(19) United States
(12) Patent Application Publication
(10) Pub. No.: US 2023/0160188 A1
(43) Pub. Date: May 25, 2023

(54) PLUMBING FIXTURE AND METHODS OF OPERATION

(71) Applicant: Angel Guard Limited, London (GB)

(72) Inventors: Jonathan Waggott, London (GB); Elaine Waggott, London (GB)

(73) Assignee: Angel Guard Limited, London (GB)

(21) Appl. No.: 17/835,451

(22) PCT Filed: Dec. 16, 2020
(86) PCT No.: PCT/EP2020/086528

§ 371 Date: Jun. 8, 2022

(30) Foreign Application Priority Data
Dec. 16, 2019 (GB) 1918463.9

(51) Int. Cl. E03C 1/126; E03C 1/05

(52) U.S. Cl. CPC E03C 1/126; E03C 1/055

(57) ABSTRACT

The present invention relates to systems and methods for reducing microbial contamination, particularly biofilm formation, in plumbing fixtures. In particular, the invention provides a computer implemented method for counteracting biofilm formation in a plumbing fixture, and a plumbing fixture in which the method may be applied. The method comprises: monitoring a parameter relating to microbial presence within the plumbing fixture, for example using a sensor; determining a level of risk related to biofilm formation based on the monitored parameter, for example based on sensor data; and in accordance with the determined level of risk, applying a countermeasure to the plumbing fixture in order to counteract biofilm formation.

### TECHNICAL FIELD

The present invention relates to systems and methods for reducing microbial contamination, particularly biofilm formation, in plumbing fixtures.

### BACKGROUND TO THE INVENTION

Plumbing fixtures are present in many locations and buildings which require a high level of sterility. For example, in hospitals sinks are provided for staff and patients to wash their hands in order to maintain a sterile environment to avoid the transfer of infections to or between patients.

However, plumbing fixtures themselves can provide a suitable environment for microorganisms to grow and biofilm to form. For example, biofilm may form within the taps or waste pipes of a sink. Such microorganisms may be difficult to detect and also difficult to remove. This means that the plumbing fixtures themselves present a source of contamination which can transfer infections to patients. Furthermore, the design of these plumbing fixtures means that when they are being used, the user may inadvertently contaminate their own clothing or a surrounding area due to splashing of waste water from the plumbing fixture. This represents a further infection risk from the plumbing fixture.

The present invention aims to overcome the problems with infection due to the design and use of known plumbing fixtures.

### SUMMARY OF THE INVENTION

At its most general, the present invention provides arrangements for reducing microbial contamination, particularly biofilm formation, in plumbing fixtures.

In a first aspect of the present invention, there is provided a computer implemented method for counteracting biofilm formation in a plumbing fixture, the method comprising: monitoring a parameter relating to microbial presence within the plumbing fixture, for example using a sensor; determining a level of risk related to biofilm formation based on the monitored parameter, for example based on sensor data; and in accordance with the determined level of risk, applying a countermeasure to the plumbing fixture in order to counteract biofilm formation. For example, the monitored parameter and level of risk may indicate that conditions within the plumbing fixture are suitable for a biofilm to begin to form, and/or that biofilm has already begun to form within or around the plumbing fixture. The method thereby allows a countermeasure to be automatically deployed in response to a risk level. In particular, as the countermeasure is applied in accordance with a determined level of risk, different countermeasures may be deployed as is considered to be appropriate. For example, a higher risk level, which may correspond to the presence of biofilm, may be related to a countermeasure suitable for eliminating any biofilm. A lower risk level, which may correspond to the detection of parameters indicative that a biofilm may soon start to form, may be related to a countermeasure suitable for pre-emptively preventing any biofilm forming. Such an arrangement may help to reduce the risk of any microorganisms becoming resistant to a particular countermeasure. The method may therefore be particularly suited for use with plumbing fixtures located in sterile areas, for example in hospitals or care facilities and the like. It is envisaged that the plumbing fixture may be a sink, or a shower, a lavatory, or any suitable plumbing fixture. In some examples, a plurality of parameters may be measured, and a level of risk associated with each parameter may be determined. The levels of risk associated with each parameter may be combined into a single level of risk (e.g. a global level of risk), wherein a countermeasure is applied in accordance with the single (or global) level of risk.

Preferably, the method may further comprise storing, for example in a computer memory, information related to any one or more of: the monitored parameter, the determined level of risk, and/or the countermeasure applied. Such information may be stored locally to the plumbing fixture, for example at a local computing system, or may be stored remotely, for example at a remote server. Storing information in this way may improve the determination of a level of risk and may also improve the selection of a countermeasure to be applied. In some embodiments, determining a level of risk may comprise analysing previously stored information. For example, determining a level of risk may comprise determining a trend in previously stored information. In particular, previously stored data includes historical information as well as recently stored information, for example the information referred to above. By analysing previously stored information it may be determined how changes in a particular parameter over time relate to biofilm formation, and so the level of risk may be determined accordingly. Additionally and/or alternatively, analysing the stored data may show correlations between a monitored parameter, a level of risk, and/or a countermeasure applied such that a level of risk may be more accurately determined in future and/or a more appropriate countermeasure applied for a determined level of risk.

Advantageously, the method may comprise a step of analysing previously stored information to determine a countermeasure to apply to the plumbing fixture. This may be particularly advantageous to ensure that any microorganisms do not develop a resistance to a countermeasure, and so countermeasures can be applied based on previous applications to reduce the chances of resistance. For example, it can be ensured that a particular countermeasure is not applied consecutively, but may be interspersed with the application of alternative countermeasures. By storing data in this way, it may also be determined that a countermeasure is particularly effective when used in response to a particular monitored parameter or level of risk, for example, and so a most effective countermeasure may be deployed in future.

Preferably, determining a level of risk may comprise comparing the monitored parameter with a predetermined threshold. For example, the threshold may be indicative of a particular risk level and so a countermeasure may be applied accordingly. In some examples, there may be multiple thresholds, wherein each threshold is indicative of a different risk level, where each risk level has a respective associated countermeasure to be applied. In some examples, the threshold may vary over time. For example, the method may comprise a step of analysing stored information in order to adjust a threshold used to determine a level of risk. In this way, the invention may provide an adaptive risk assessment, for example based on machine learning. For example, a threshold for a first parameter may be changed over time according to the monitoring of a second parameter if monitoring the second parameter demonstrates that the first parameter is not as sensitive to biofilm formation or contamination as initially assumed.

Advantageously, the method may further comprise receiving a level of risk related to biofilm formation associated with another plumbing fixture; and applying a countermeasure to the plumbing fixture in accordance with the received level of risk. In this way, the method may allow for a network of plumbing fixtures to be monitored simultaneously with risk levels being shared between plumbing fixtures, wherein each plumbing fixture is able to automatically apply a countermeasure if a received risk level necessitates it. This may be particularly advantageous to inhibit or prevent microorganisms spreading between plumbing fixtures. For example, if a monitored parameter indicates a high level of risk in a first plumbing fixture, then nearby plumbing fixtures may be made aware of the nearby risk and apply a countermeasure to inhibit spread of a microorganism to the nearby plumbing fixtures. The risk level may be shared directly between plumbing fixtures, or may be received at a plumbing fixture from a central (e.g. remote) computing system, such as a server, for example. Similarly, the method may comprise receiving an instruction to apply a countermeasure to the plumbing fixture; and applying a countermeasure to the plumbing fixture in accordance with the instruction. In this way, a plumbing fixture may be remotely controlled to apply a countermeasure if it is determined that doing so may help reduce the spread of biofilm or microorganisms through a group of plumbing fixtures. The instruction may be received from another plumbing fixture or from a central (e.g. remote) computing system, such as a server, for example.

Advantageously, the method may further comprise a step of adjusting a monitoring frequency, for example by pausing monitoring for a period of time. Preferably, the monitoring frequency is determined according to the determined level of risk. In some embodiments, pausing may be for a variable time period, which may vary depending on the determined risk level. For example, if a level of risk is low, then it may not be necessary to monitor a parameter as frequently as when a risk level is high. By adjusting the monitoring frequency of a parameter in this way, the method may thereby be made more efficient as monitoring may not need to be continuously performed, and may maintain responsiveness to perceived threats if a risk level is high. In an embodiment, the monitoring frequency may be fixed, and monitoring may be paused for a fixed time period which may be set by an operator of the system, for example. The monitoring frequency, and duration of a pause may also be dependent on the monitored parameter. For example, a water temperature may be monitored more regularly than a biofilm sensor. In some examples, a monitoring frequency and pause duration may be dependent on a risk level which has been received from another plumbing fixture. In particular, if a received risk level is high then the duration of the pause may be reduced such that the monitoring frequency is increased. In some examples, adjusting the monitoring frequency may comprise turning off monitoring entirely, or pausing monitoring indefinitely.

Optionally, monitoring a parameter may comprise monitoring any one or more of: a hot water temperature, a cold water temperature, a water flow rate, and/or a biofilm level. For example, a level of risk may be determined for each monitored parameter, and the levels of risk may be combined into a single level of risk. For example, the highest level of risk determined for any parameter may be used as the single level of risk.

Preferably, applying a countermeasure may comprise any one or more of: flushing water through the plumbing fixture, performing a disinfection, introducing metal ions to the water supply and/or disabling the plumbing fixture. Advantageously, performing a disinfection may comprise any one or more of: performing ultraviolet (UV) light disinfection (that is, using UV-frequency light to disinfect the plumbing fixture), performing an ozone disinfection (that is, using ozone gas to disinfect the plumbing fixture), and/or using any other suitable disinfection means. For example, a chemical disinfection may comprise flushing a chlorine-based chemical disinfectant through the plumbing fixture. In some embodiments, flushing water through the plumbing fixture may be performed with water having a temperature of at least 37° C., preferably at least 50° C., or at least 61° C., such as 70° C. In some embodiments, the countermeasure may comprise holding water within the plumbing fixture for a predetermined period of time. For example, water at 70° C. may be held within the plumbing fixture for 3 minutes; water at 60° C. may be held within the plumbing fixture for 5 minutes; or water at 55° C. may be held within the plumbing fixture for 10 minutes. Disabling the plumbing fixture may comprise shutting off a water supply to the plumbing fixture so that it cannot be used, for example. In other examples, disabling the plumbing fixture may comprise activating an audible or visible alert, such as an alarm, a light, or an alert on a screen, which may warn a user not to make use of the plumbing fixture. Preferably, any means which may prevent or dissuade a user from using the plumbing fixture may be considered as means for disabling the plumbing fixture as a countermeasure to counteract biofilm formation.

Optionally, the monitoring frequency, and the duration of a pause in monitoring, may be determined according to a countermeasure which has been applied. For example, a pause after a water flush may have a shorter duration than a pause after a chemical disinfection.

According to a second aspect of the invention, there is provided a system for counteracting biofilm formation in a plumbing fixture, the system comprising: a plumbing fixture; a local computing system; and a remote computing system, for example a remote server, wherein the local computing system and the remote computing system are configured to perform a method according to the first aspect of the invention. In particular, the method steps may be divided between the local computing system and the remote computing system in any suitable manner. For example, the local computing system may be configured to monitor a parameter and apply a countermeasure and the remote computing system may be configured to determine a level of risk related to biofilm formation based on the monitored parameter.

According to a third aspect of the present invention, there is provided a computer implemented method for counteracting biofilm formation in a plurality of plumbing fixtures, the method comprising: monitoring, for each plumbing fixture, a parameter relating to microbial presence within that plumbing fixture, for example with a sensor; determining, for each plumbing fixture, a level of risk related to biofilm formation based on the monitored parameter for that plumbing fixture, for example based on sensor data; and applying, for each plumbing fixture, a countermeasure in accordance with the determined level of risk for that plumbing fixture in order to counteract biofilm formation. For example, for each plumbing fixture the method may comprise substantially the same method steps as described above in relation to the first aspect of the invention. In this way, the method of the third aspect of the present invention allows independent countermeasure control in a plurality of plumbing fixtures.

Preferably, the plurality of plumbing fixtures may be divided into a plurality of groups, and the method may further comprise, in accordance with the determined level of risk for a first plumbing fixture, applying a countermeasure to each plumbing fixture within a group to which the first plumbing fixture belongs in order to counteract biofilm formation. By providing groups of plumbing fixtures in this way, and applying countermeasures to all plumbing fixtures within a particular group, the present invention may help to ensure that microorganisms do not spread to other plumbing fixtures within a given group. In some examples, each group may have an associated location identifier, wherein the level of risk related to biofilm formation for each plumbing fixture may be determined according to the associated location identifier for the group to which each plumbing fixture belongs. For example, it may be desirable to apply a stricter standard of cleanliness to plumbing fixtures installed at one location, and so by taking a location identifier into account a risk level may be adjusted accordingly to apply the strict standard. For example, where plumbing fixtures are present in a hospital, it may be desirable to enforce a stricter standard of cleanliness in surgical areas than in non-surgical areas (e.g. an outpatient waiting area)—that is, a risk level in a surgical area may be higher than a non-surgical area for the same monitored parameter. A location identifier may therefore be associated with plumbing fixtures in a surgical area to ensure that the risk level is determined according to the stricter standard.

According to a fourth aspect of the present invention, there is provided a system for counteracting biofilm formation in a plurality of plumbing fixtures, the system comprising: a plurality of plumbing fixtures; a plurality of local computing systems, wherein each local computing system is associated with a respective plumbing fixture; and a remote computing system, such as a server, associated with the plurality of plumbing fixtures, wherein the plurality of local computing systems and the remote computing system are configured to perform a method according to the first and/or the third aspect of the present invention. For example, the method steps may be divided between the local computing system of each plumbing fixture and the remote computing system, and the steps may be divided in any suitable manner. In some examples, the remote computing system may store and analyse data, and send a determined risk level to the local computing system which directly operates a plumbing fixture.

According to a fifth aspect of the present invention, there is provided a plumbing fixture for reducing contamination, the plumbing fixture comprising: a basin affixable to a mounting surface at a proximal end; a water inlet through which a stream of water may be introduced to the basin; and a drain to allow water to flow out of the basin; wherein the basin is an enclosure having am opening at a distal end to allow a user to access the stream of water, the water inlet, being formed in an upper surface of the basin, and wherein the water inlet and the basin are configured such that the entire stream of water is incident on an interior surface of the basin, wherein the interior surface is angled to reflect the entire stream of water away from the opening. In this way, the fifth aspect of the present invention provides a plumbing fixture, for example a sink, which reduces splashing of water (in particular, egress of water through the basin opening) and so reduces contamination by microorganisms, in particular contamination of a user or of the environment surrounding the plumbing fixture. The plumbing fixture may therefore be particularly suited for use in sterile areas, for example in hospitals or care facilities and the like.

Preferably, the basin is generally tubular and has a longitudinal axis, and wherein the basin is disposed such that the longitudinal axis is at an acute angle with respect to a vertical plane. Preferably, the longitudinal axis may be at an angle of between 50° and 70° with respect to a vertical plane. For example, the basin may have any suitable cross-sectional shaped perpendicular to its longitudinal axis, though a cylindrical basin may be particularly preferable. By providing the basin as a tubular structure and at an angle in this way, the basin is particularly effective at reducing splashing of water and ensuring water is contained within the basin itself. In particular, the angle of the basin may provide an integrated lip or rim which helps to prevent egress of water through the opening.

Advantageously, the plumbing fixture may comprise a tap for providing the stream of water, wherein the tap is spaced away from the water inlet. By spacing the tap away from the water inlet in this way, the direct transfer of microorganisms between the tap and the basin in minimised, preventing contamination of the basin and/or of the tap and reducing a risk of biofilm formation. This may be particularly advantageous when cleaning the basin, as spacing the tap away from the water inlet helps to ensure that no microorganisms are transferred from the cloth used to wash the basin to the tap. In some examples, the tap may have a proximal end and a distal end, wherein the distal end of the tap may be disposed below the proximal end. This arrangement may ensure that when the plumbing fixture is not in use there is no standing water within the tap and so biofilm build-up within the tap may be prevented or minimised.

Optionally, the tap may be configured to produce a generally planar stream of water. A planar stream of water may minimise splashing of water, such that egress of water through the basin opening is minimised. A planar stream of water is also highly directional (e.g. coherent), and so it can be ensured that when the stream of water is incident on an interior surface of the basin all the water is reflected away from the basin opening.

Preferably, the basin may protrude from a mounting surface, and may further comprise a housing for the tap, wherein the housing has a curved upper exterior surface. In this way a user cannot access the tap, and so the transfer of microorganisms between a user and the tap is prevented. In addition, as the housing has a curved upper surface a user cannot rest foreign objections on the housing, which further reduces the risk of contamination of the plumbing fixture as foreign objects may not be sterile. Preferably, the basin and the housing may be made of materials which are chosen to reduce contamination and which are easy to clean, for example glass.

Advantageously, the plumbing fixture may further comprise an air extraction system which is configured to generate a negative pressure within the basin. By ‘negative pressure’ it should be understood that the resulting pressure within the basin is less than atmospheric pressure, to ensure that air preferentially flows into the basin and not out of the basin. By providing an air extraction system in this way, it can be ensured that aerosols and aerosolised particles do not escape the basin when the basin is in use, which may help prevent the spread of air-borne pathogens. Preferably, the air extraction system may be configured to operate only when the plumbing fixture is in use. For example, the air extraction system may comprise an extractor fan connected to an air duct having an outlet in the basin, the extractor fan being configured to generate an air flow, drawing air out of the basin through the outlet when in use. Aerosols can be defined as liquid or solid particles suspended in the air. Bic-aerosols are aerosols consisting of particles of any kind of organism. The characteristics of bio-aerosols differ depending on environmental influences such as humidity, air flow, and temperature. Aerosols, which are responsible for the transmission of airborne micro-organisms by air, consist of small particles named droplet nuclei (1-5 μm) or droplets (>5 μm). Droplet nuclei can stay airborne for hours, transport over long distances and contaminate surfaces by falling down. In a review article from 2006 (Tang, Li, Eames, Chan, & Ridgway, 2006), the authors found for SARS-CoV-1 that “particles of diameters 1-3 μm remained suspended almost indefinitely, 10 μm took 17 min, 20 μm took 4 min, and 100 μm took 10 seconds to fall to the floor” (Tang et al., 2006). This article notes that aerosol transmission is a well-known and important exposure pathway for infectious agents such as influenza and other viruses including coronaviruses. As discussed in this article, SARS-CoV-1 viral RNA was found in air samples, and long-range aerosol transport was implicated as the cause of the spread of the disease in several studies. It has been proven that droplets can contaminate surfaces in a range of over 2 meters. The droplets are also capable of penetrating deep into the lungs, offering a potential route of infection. The susceptibility of acquiring an infectious agent is determined by factors such as: virulence; dose; and pathogenicity of the micro-organism; and the host's immune response. Bio-aerosols can contain pathogens including influenza, Mycobacterium tuberculosis, Staphylococcus aureus, Varicella Zoster Virus, Streptococcus spp. or Aspergillus spp. Moreover, bio-aerosols can be generated by devices such as ventilation systems, showers, taps and toilets. Showers and tap water are also able to spread environmental microbes such as Legionella spp. Thus, the air extraction system can ensure that such microbes are not able to spread. Preferably, the air extraction system may comprise a filter device configured to remove aerosol particles from a flow of air removed from the basin. For example, the filtering device may comprise an air filter, such as a medical air filter. The filtering device may ensure that aerosols and aerosolised particles are trapped, and therefore do not spread either around the plumbing fixture itself or around an air outlet where air may be extracted to by the extraction system. Preferably, the filtering device may be configured to remove aerosol particles having a diameter of at least 1 μm, though of course it is envisaged that smaller particles may also be removed.

In some examples, the plumbing fixture may comprise any one or more of: a biofilm sensor, for example within the tap; a flow sensor at the water inlet; a flow sensor at the drain; and/or a water temperature sensor. For example, these sensors may be used to monitor parameters for determination of a risk level. Optionally, the plumbing fixture may comprise a countermeasure device, for example a system for adding metal ions to a water supply, ultraviolet (UV) lamps for ultraviolet sterilisation, a source of ozone gas, and/or a system for performing a chemical disinfection. Advantageously, the plumbing fixture may further comprise a computing system which is configured to control operation of the plumbing fixture. In particular, the computing system may be configured to carry out a method as described above with respect to the first aspect of the present invention. Optionally, the computing system may be configured to control the flow and temperature of water to the basin through the water inlet. This may allow the plumbing fixture to function automatically, for example when a user is detected. This may reduce contamination by ensuring that a proper handwashing procedure is followed by a user. For example, the computing system may control the stream of water into the basin, for example by automatically turning the stream of water off and on to perform an automatic handwashing routine. The computing system may also control the temperature of water delivered into the basin, for example by independently adjusting flow rates of hot and cold water to the plumbing fixture.

Preferably, the plumbing fixture may comprise a radio-frequency identification (RFID) sensor. In this way, the plumbing fixture may be configured to identify a user, and so the sink may be able to track usage patterns and data related to how users interact with the plumbing fixture. In particular, by comprising an RFID and a computing system for controlling the plumbing fixture, the plumbing fixture is able to ensure that every user follows proper handwashing protocol, and is able to track each users' compliance with such a protocol. An RFID sensor may be used to identify the last user of a sink before a blockage or other problem is identified, for example.

In some examples, the plumbing fixture may comprise a proximity sensor to determine if a user is close to the plumbing fixture. For example, the proximity sensor may be used to stop a countermeasure being applied if a user is nearby in order to minimise risk to a user, e.g. a risk of contamination. In some embodiments, the proximity sensor may be configured to activate an alert if a user is nearby, for example.

### BRIEF DESCRIPTION OF THE DRAWINGS

An embodiment of the invention is discussed below in more detail with reference to the accompanying drawings, in which:

FIG. 1 is a flow diagram showing a computer implemented method for counteracting biofilm formation in a plumbing fixture according to an embodiment of the present invention;

FIG. 2 is a flow diagram showing a computer implemented method for counteracting biofilm formation in a plurality of plumbing fixtures according to an embodiment of the present invention;

FIG. 3 is cross-sectional view of a plumbing fixture which is an embodiment of the present invention;

FIG. 4 is a front view of a plumbing fixture which is an embodiment of the present invention;

FIG. 5 is a schematic diagram showing the components of a plumbing fixture in an embodiment of the present invention;

FIG. 6 is a cross-section view of a plumbing fixture which is a further embodiment of the present invention; and

FIG. 7 depicts an example computing system.

### DETAILED DESCRIPTION; FURTHER OPTIONS AND PREFERENCES

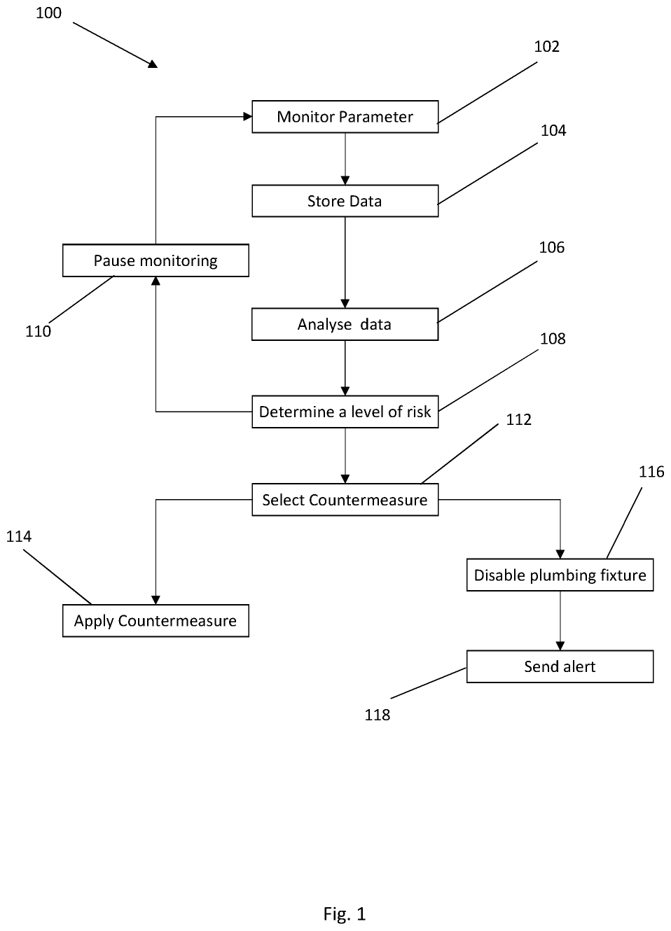

FIG. 1 is a flow diagram showing a computer implemented method 100 for counteracting biofilm formation in a plumbing fixture according to an embodiment of the present invention. The method 100 may be applied to any suitable plumbing fixture, such as a sink, a bath, a water closet (WC) bowl or flush toilet, or a shower, and may be carried out using a processing system (i.e. a local computing system) which is present locally to the plumbing fixture and/or a remote processing system (i.e. a remote computing system) such as a server. In some examples, the method 100 may be used with respect to multiple plumbing fixtures simultaneously with a central server overseeing operation of each plumbing fixture.

In a first step, the method 100 comprises monitoring a parameter 102 relating to microbial presence within the plumbing fixture. For example, monitoring a parameter 102 may comprise monitoring a water temperature, a water flow rate and/or a biofilm level at various locations within the plumbing fixture. Of course, it is considered that any parameter which may affect microorganism growth within the plumbing fixture may be monitored in accordance with the present invention.

The data which is collected by monitoring a parameter 102 may then be stored at step 104. For example, data may be stored at a local processing system and/or at a remote processing system such as a remote server. As will be explained in more detail below, by storing data in this way a risk level may be determined based at least in part on an analysis of historical data which has been previously stored. Of course, it will be appreciated that it is not only data relating to monitored parameters which may be stored, but information relating to a determined risk level, and/or a countermeasure applied may also be stored in accordance with the present invention.

After data has been stored, the data may be analysed 106. For example, analysing data may comprise determining a trend in a monitored parameter and/or may comprise comparing a monitored parameter with a threshold level. This analysis is then used to determine a level of risk 108 related to biofilm formation based on the monitored parameter.

For example, if the monitored parameter is a hot water temperature, a drop of the hot water temperature below a first threshold temperature (e.g. below 55° C.) may indicate a first, low risk level and a drop of the hot water temperature below a second, lower threshold temperature (e.g. below 50° C.) may indicate a second, higher risk level. In this example, the higher risk level is indicative that the hot water temperature presents a suitable environment for biofilm formation, for example. Additional risk levels may be defined in a similar way, and the temperature levels and associated risk levels may be preset by an operator of the system, for example. Similar considerations may be made for a low water temperature, in some embodiments.

Additionally or alternatively, where the monitored parameter is a hot water temperature, then the risk level may be indicated by the magnitude of a temperature deviation from an average, or normal level (which is calculated from stored historical data, for example, or may be input by an operator of the system). For example, if the hot water temperature is normally below 55° C. but above 50° C. then a deviation of more than 3° C. from the average level may indicate a first, low risk level. If the hot water temperature is normally below 50° C. but above 45° C. then a deviation of more than 3° C. from the average level may indicate a second, higher risk level. Additional risk levels may be defined in a similar way, and the temperature levels and associated risk levels may be preset by an operator of the system, for example. Similar considerations may be made for a low water temperature, in some embodiments.

In some embodiments, the monitored parameter may be the amount of biofilm formation detected by a biofilm sensor, and so the level of risk may be directly related to an amount of biofilm which has built up within the plumbing fixture. In some examples, the determined level of risk may also be stored, for example locally or at a remote server, to allow tracking of the level of risk related to the plumbing fixture over time.

In some examples, determining a level of risk 108 may also comprise analysing information related to previously determined levels of risk. For example, if a low level of risk is determined repeatedly within a short time span then this may indicate a longstanding problem within the plumbing fixture and so the level of risk may be increased accordingly.

In accordance with the determined level of risk, a monitoring frequency may be adjusted. In particular, monitoring may be paused 110 for a period of time. For example, if a determined level of risk is low, then monitoring may be paused for six hours to reduce the monitoring frequency, and if a determined level of risk is high then monitoring may be paused for thirty minutes to increase the monitoring frequency. Of course, the monitoring frequency and duration of the pause may be determined by an operator of the system in which the method 100 is run.

In accordance with the determined level of risk, a countermeasure is selected 112. In some embodiments, information about the selected countermeasure may be stored, for example locally or at a remote server, such that the selection of a countermeasure may be based at least in part on a previously applied countermeasure. For example, selecting a countermeasure 112 may comprise selecting based only on the determined level of risk, or information relating to previously applied countermeasures may also be taken into account. For example, a countermeasure may be applied if it is particularly effective for reducing a threat level or may be avoided to reduce the risk of microorganisms developing resistance to that particular countermeasure.

For example, a countermeasure may comprise any one or more of: flushing water through the plumbing fixture, particularly hot water; performing a chemical disinfection of the plumbing fixture, which may be performed automatically by the plumbing fixture or may be applied manually, for example by a maintenance professional; introducing metal ions to the water supply to the plumbing fixture; using UV-frequency light to disinfect the plumbing fixture; using ozone to disinfect the plumbing fixture; using any other suitable disinfection means; and/or disabling the plumbing fixture 116. For example, a chemical disinfection may comprise flushing a chlorine-based chemical disinfectant through the plumbing fixture. In some embodiments, flushing water through the plumbing fixture may be performed with water having a temperature of at least 37° C., preferably at least 50° C., or at least 61° C., such as 70° C. In some embodiments, the countermeasure may comprise holding water within the plumbing fixture for a predetermined period of time. For example, water at 70° C. may be held within the plumbing fixture for 3 minutes; water at 60° C. may be held within the plumbing fixture for 5 minutes; or water at 55° C. may be held within the plumbing fixture for 10 minutes. Disabling the plumbing fixture may comprise shutting off a water supply to the plumbing fixture so that it cannot be used, for example. In other examples, disabling the plumbing fixture may comprise activating an audible or visible alert, such as an alarm, a light, or an alert on a screen, which may warn a user not to make use of the plumbing fixture. Preferably, any means which may prevent or dissuade a user from using the plumbing fixture may be considered as means for disabling the plumbing fixture as a countermeasure to counteract biofilm formation. In some examples, the countermeasure which is applied may also affect the monitoring frequency, and in particular the duration for which monitoring is paused 110. For example, if an applied countermeasure is a water flush then the duration of the pause may be shorter than if the applied countermeasure is a chemical disinfection, as a chemical disinfection may generally be expected to be more effective at removing or reducing biofilm.

In particular, where the determined risk level is low, a countermeasure is applied 114, such as flushing water through the plumbing fixture. In some examples, a low level of risk may not require any countermeasure to be applied to the plumbing fixture, and so the only action taken is to pause monitoring 110. Where the determined risk level is high, in addition to and/or as an alternative to applying a countermeasure 114 such as a chemical disinfection, the plumbing fixture may be disabled 116. When a plumbing fixture is disabled, an alert may be sent 118 to a central server to inform an operator that a high risk level has been determined in the plumbing fixture which may require manual disinfection. In such cases a risk level or countermeasure instruction may be sent to other plumbing fixtures in order to inhibit or prevent the spread of contamination between plumbing fixtures.

By way of example, the method 100 may determine a level of risk 108 which may be selected from four risk levels —level 1, level 2, level 3 or level 4, wherein level 1 represents a lowest risk of biofilm formation and level 4 represents a highest risk of biofilm contamination, or that an amount of biofilm has been detected. For example, if the determined level of risk is level 1 or level 2, the countermeasure which is applied may comprise flushing water (for example, hot water or a mixture of hot and cold water) through the plumbing fixture. If the determined level of risk is level 3, the countermeasure which is applied may be a chemical disinfection. Where a chemical disinfection is performed, users may be alerted not to use the plumbing fixture while the disinfection is underway. If the determined level of risk is level 4, then the countermeasure may be to disable the plumbing fixture. Disabling a plumbing fixture may comprise shutting off a water supply to the plumbing fixture so that it cannot be used, for example. In other examples, disabling the plumbing fixture may comprise activating an audible or visible alert, such as an alarm, a light, or an alert on a screen, which may warn a user not to make use of the plumbing fixture. Preferably, any means which may prevent or dissuade a user from using the plumbing fixture may be considered as means for disabling the plumbing fixture as a countermeasure to counteract biofilm formation.

The level of risk which is determined may be stored in a memory (e.g. at a local or remote computing system), and the determined level of risk for the plumbing fixture may be tracked over time such that if a risk level frequently occurs, or countermeasures are frequently applied to the plumbing fixture, the plumbing fixture may be disabled for manual assessment as frequent risk level alerts may be indicative of a longstanding problem or something which cannot be addressed by the usual countermeasures.

As an example of changing subsequent applied countermeasures, if a first level of risk is determined to be level 3 then a chemical disinfection is applied. If a second level of risk determined at a later time to the first level of risk is also level 3, then a water flush may be applied as the countermeasure. This may ensure microorganisms do not develop a resistance to the chemical disinfection. This cycle may be repeated for further determined risk levels at later times. In particular, the system may be calibrated such that a countermeasure is not repeated if it has been performed previously within a predetermined time period. For example, a countermeasure may not be applied if it has been performed within the preceding 30 days.

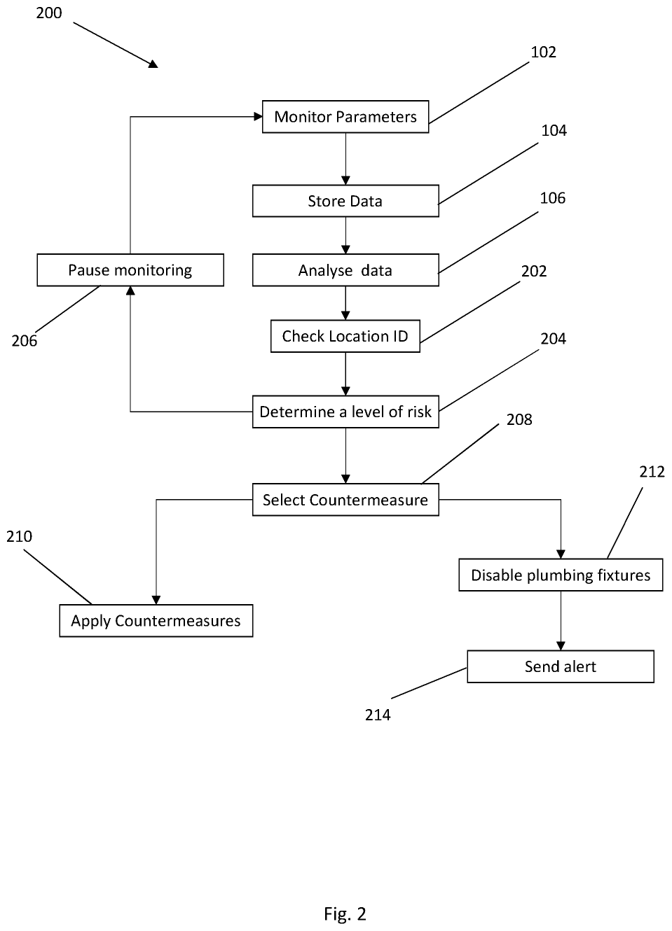

FIG. 2 is a flow diagram of a method 200 for counteracting biofilm formation in a plurality of plumbing fixtures according to an embodiment of the present invention. Steps which are the same as those described above with respect to FIG. 1 are given the same reference numerals, though it will be appreciated that, in this embodiment, the steps of monitoring parameters 102, storing data 104 and analysing stored data 106 relate to a plurality of plumbing fixtures and not a single plumbing fixture. That is, parameters are measured at multiple plumbing fixtures, data is stored for multiple plumbing fixtures, and data is analysed for multiple plumbing fixtures. These steps, and the other steps of the method 200, may be carried out locally to each plumbing fixture, for example at a local processing system within each plumbing fixture, or may be carried out at a remote server, for example.

The plurality of plumbing fixtures are preferably divided into a number of groups. In particular, these groups may be associated with a location of the plumbing fixtures, for example a particular location within a hospital (such as a ward) or care facility. Each plumbing fixture may therefore by associated with a particular location identifier (location ID). After the data has been analysed, a location ID of each plumbing fixture may be checked 202, and the location ID may influence the level of risk which is determined 204 for each pluming fixture. For example, if a plumbing fixture is located within a surgical area of a hospital, it may result in a higher risk level being assigned than for a plumbing fixture located in a non-surgical area. The determination of a level of risk may also depend on previously stored data for each plumbing fixture, such as a historical parameter and/or risk level data in substantially the same manner as described above with respect to FIG. 1.

In a similar manner as has been described above, monitoring may be paused 206 so as to adjust the monitoring frequency for any plumbing fixture within the plurality of plumbing fixtures according to the determined level of risk. This may include pausing monitoring for all plumbing fixtures within the plurality of plumbing fixtures, or for all plumbing fixtures within a group (that is, having the same location ID). For example, it a determined level of risk is low, then monitoring may be paused for six hours to reduce the monitoring frequency, and if a determined level of risk is high then monitoring may be paused for thirty minutes to increase the monitoring frequency. That is, the monitoring frequency and duration of the pause may be dependent on the level of risk. Of course, the duration of the pause may be determined by an operator of the system in which the method 200 is run.

In accordance with the level of risk determined for each plumbing fixture, a countermeasure is selected 208. In some embodiments, information about the selected countermeasure may be stored, for example locally or at a remote server, such that the selection of a countermeasure may be based at least in part on a previously applied countermeasure. For example, selecting a countermeasure 208 may comprise selecting based only on the determined level of risk, or information relating to previously applied countermeasures may also be taken into account. For example, a countermeasure may be applied if it is particularly effective for reducing a threat level or may be avoided to reduce the risk of microorganisms developing resistance to that particular countermeasure.

Applying a countermeasure 210 and/or disabling a plumbing fixture may be substantially as described above in relation to FIG. 1. However, in addition, applying a countermeasure 210 and/or disabling a plumbing fixture 212 may comprise applying the countermeasure to and/or disabling each plumbing fixture within the plurality of plumbing fixtures or each plumbing fixture with a same location ID. When a plumbing fixture is disabled, an alert is sent 214. For example, the alert may be sent to a central server and/or to nearby plumbing fixtures to alert them that a high risk level has been determined. The central server may then decide whether to take further action, such as alerting other plumbing fixtures. Alerting other plumbing fixtures may mean that they implement their own countermeasures in response to the alert, which may be dependent on a risk level associated with the alert.

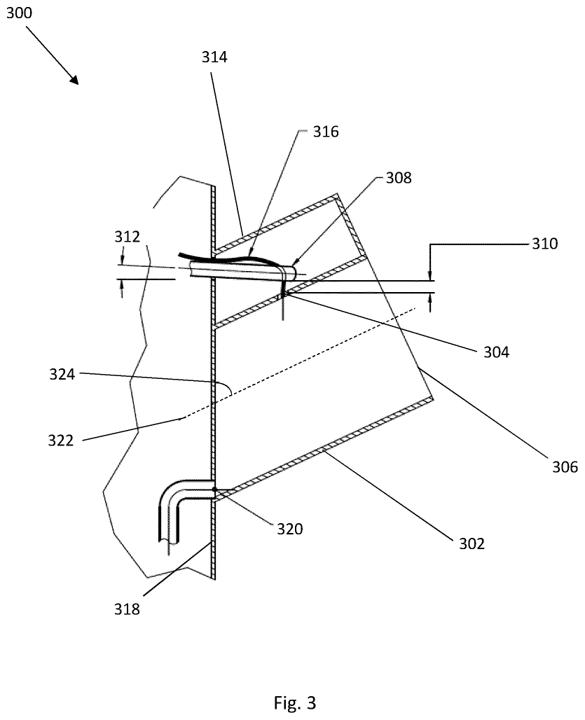

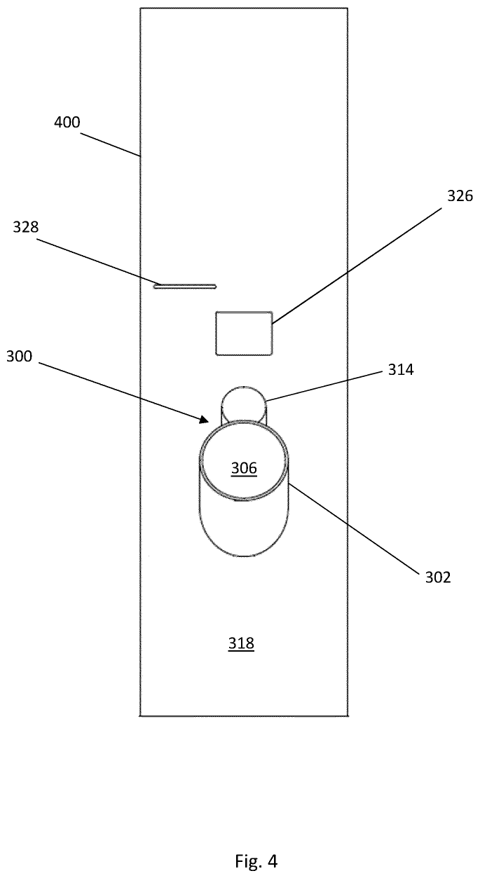

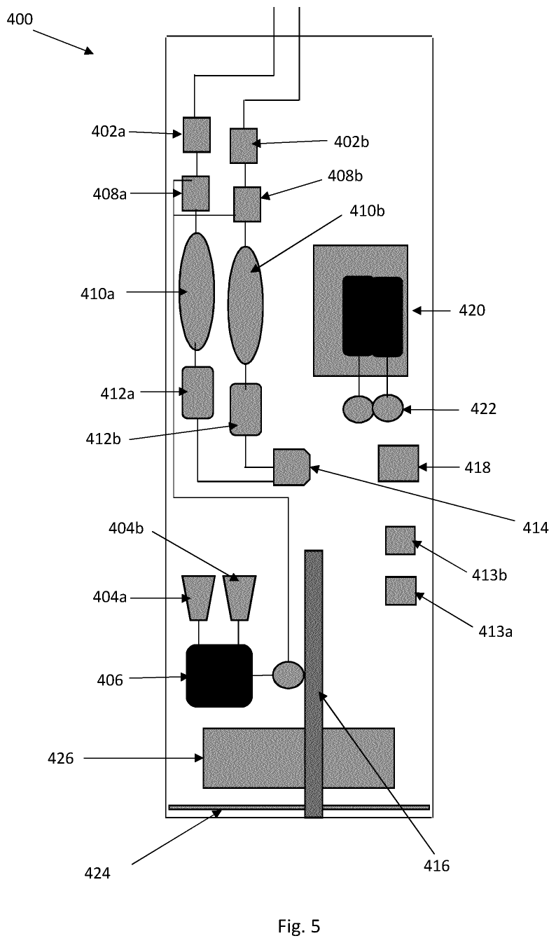

FIG. 3 shows a cross-section view of a plumbing fixture 300 which is an embodiment of the present invention. In particular, the plumbing fixture 300 is a sink which is adapted to reduce contamination. FIG. 4 shows a front view of the plumbing fixture 300. In particular, the plumbing fixture 300 is mounted to an enclosure 400, the contents of which are discussed in more detail below with respect to FIG. 5, such that a front surface of the enclosure provides a mounting surface 318 for the plumbing fixture 300.

The plumbing fixture comprises a basin 302 which is a generally cylindrical enclosure having an opening 306 at a distal end which is closest to a user, the opening 306 allowing a user to place their hands inside the basin 302 for washing. The basin 302 protrudes from a mounting surface 318, where the basin 302 is mounted at a distal end of the basin 302. In some examples mounting surface 318 may be a wall of a building, for example. However, as shown in FIG. 4 the mounting surface 318 is the front face of an enclosure which contains components for the plumbing fixture 300, as shown in FIG. 5 discussed below. In particular, a longitudinal axis 322 of the basin 302 is disposed at an acute angle 324 with respect to a vertical plane, for example the mounting surface 318 as shown in FIG. 3. For example, the angle 324 may be between 50° and 70°. In this way, the distal end of the basin 302 is vertically higher than the proximal end of the basin 302. This arrangement minimises egress of water through the opening 306. The basin may be made of any material which is easy to clean, but in a particularly preferred example the basin is made of glass.

In an upper surface of the basin 302 there is provided a water inlet 304 through which a stream of water is introduced into the basin 302 when in use. In particular, the stream of water is introduced into the basin 302 from a tap or faucet 308 which produces a generally planar stream of water. It will be appreciated that due to the angle of the basin 302, the generally planar stream of water provided through the opening 304 is incident on an interior surface of the basin 332 to be reflected away from the opening 306 and thus away from the user. In this way, the plumbing fixture 300 is configured to minimise splashing, that is, egress of water from the basin opening 306, where splashing of water out of the opening 306 would otherwise lead to contamination, e.g. of a user and/or of an area surrounding the plumbing fixture 300 due to the spread of waste water which may contain microorganisms.

It will be appreciated from FIG. 3 that the water inlet 304 is set back from the opening 306 at an appropriate distance to ensure that egress of water through the opening 306 directly from the water inlet 304 is prevented. In addition, the water inlet 304 is set in a proximal half of the basin 302 such that a user has to fully insert their hands and at least a portion of their forearms into the basin for washing, which further aids prevention of water passing through the opening 306.

The tap 308 is vertically spaced away from the water inlet 304 such that there is a gap 310 between the tap 308 and the water inlet 304. For example, the distance 310 may be at least 20 mm to ensure that an air gap between the tap 308 and the water inlet 304 is sufficient to minimise or eliminate transfer of microorganisms between the basin 302 and the tap 308 through the water inlet 304. Furthermore, the distance 310 ensures that when the basin 302 is being cleaned, a cleaning cloth does not transfer microorganisms from the cloth to the tap 308. This is particularly important as the tap 308 is provided in a housing 314 and so cannot be easily cleaned if contamination does occur.

Although not shown in FIG. 3, the basin 300 comprises a biofilm sensor and a water flow rate sensor within or on the outside of the tap 308. For example, the biofilm sensor may comprise a SOLmicroTEK™ biofilm sensor.

These sensors allow the plumbing fixture 300 to be used in a method as described above with respect to FIGS. 1 and 2, to enable monitoring of parameters relating to microbial presence and hence the determination of a level of risk related to biofilm formation within the plumbing fixture 300. In addition, a flow rate sensor within the tap 308 may be used in conjunction with a flow rate sensor within the drain 320 to determine how much water is used by the plumbing device 300 and also to detect if a user is pouring additional liquids into the basin 302 for disposal, which may be a contamination risk, and/or to determine if the drain 320 is blocked to prevent waste water leaving the basin 302. In response to detection of such activity, the plumbing fixture 300 may apply a countermeasure substantially as described above with respect to FIGS. 1 and 2. In some embodiments, the drain may comprise a valve which may be closed, manually and/or by a processing system (see below). The valve may form part of a countermeasure system. For example, the valve may be closed in order to retain a chemical disinfectant and/or water within the basin 302 and the drain 320 for a predetermined period of time in order to disinfect the plumbing fixture 300 and/or remove biofilm. This valve may be positioned in a waste pipe of the plumbing fixture 300 as discussed below.

As shown in FIG. 4, an upper surface of the housing 314 is curved to ensure that a user cannot rest any foreign objects on top of the housing 314 or the basin 302, which reduces the risk of contamination of the plumbing fixture 300 from foreign objects which may not be sterile. The housing 314 is preferably made from the same material as the basin, preferably glass.

In addition to being spaced away from the water inlet 304, the tap 308 is also disposed at an angle 312; in particular a distal end of the tap 308 is disposed vertically below the proximal end. By angling the tap 308 in this way, it can be ensured that no standing water is present within the tap 308 when the plumbing fixture 300 is not in use. This helps to ensure that the tap 308 provides an environment which is hostile to biofilm formation.

A tube 316 is also present in the housing 314 through which hand gel and/or soap may be introduced to the basin through the water inlet 304.

The plumbing fixture 303 further comprises a drain 320 through which waste water passes from the basin 302 to a plumbing system in the building in which the plumbing fixture is installed. As noted above, the drain 320 may comprise a flow rate sensor to detect any liquid which is disposed of by a user, which may present a contamination risk, and a valve which may be closed in order to retain a chemical disinfectant and/or water within the basin 302 and the drain 32C for a predetermined period of time in order to disinfect the plumbing fixture 300 and/or remove biofilm. This valve may be positioned in a waste pipe of the plumbing fixture 300 as discussed below.

The mounting surface 318 additionally comprises a screen 326 which may be used to display alerts to a user (for example, to inform a user that the plumbing fixture 300 is disabled, or when a countermeasure is being applied) and/or to instruct a user how to interact with the plumbing fixture 300 to wash their hands. For example, the screen 326 may display information relating to the current water flow (temperature, remaining duration of water flow etc.), show videos to a user demonstrating proper handwashing technique and the like. The screen 326 may also show a countdown clock to help users to time each part of a handwash procedure (e.g. rinsing, applying soap, lathering, scrubbing, drying) to ensure that it is performed correctly. In some examples the screen 326 may be used to display alerts to users informing them that the plumbing fixture 300 is currently disabled, for example in response to a high risk level or while a countermeasure is being applied. The mounting surface 318 also comprises a paper towel dispenser 328 which may be used to provide a user with paper towels to dry their hands with after using the plumbing fixture.

FIG. 5 is a schematic diagram showing the components of a plumbing fixture, such as a plumbing fixture 300 discussed above with respect to FIGS. 3 and 4. In particular, FIG. 5 shows the contents of an enclosure 400 to which the plumbing fixture 300 may be mounted. As shown in FIG. 5, water enters the enclosure 400 at the top and leaves the enclosure 400 at the bottom. All of the pipes within the enclosure 400 are preferably made of materials having antibacterial or antimicrobial properties, for example the pipes may have an antimicrobial coating.

Water into the plumbing fixture is controlled by valves 402a and 402b. In particular, valve 402a controls the supply of hot water and valve 402b controls the supply of cold water. A thermometer may be located proximate to each valve 402a, 402b to monitor the incoming hot and cold water temperatures, which may be used in a method as described above in relation to FIGS. 1 and 2. The hot and cold water pipes may additionally and/or alternatively comprise a biofilm sensor for monitoring a biofilm level, for example proximate to the valves 402a, 402b. Data collected by the biofilm sensors may be used in a method as described above with respect to FIGS. 1 and 2. For example, the biofilm sensor may comprise an electronic biofilm sensor, such as a SOLmicroTEK™ biofilm sensor.

The plumbing fixture comprises a number of countermeasure devices. For example, a first countermeasure device is provided to enable a chemical disinfection of the plumbing fixture when required. The first countermeasure device comprises two chemical holding tanks 404a, 404b, which are connected to a mixing chamber 406 where the chemicals are mixed before being delivered into the hot and cold water supply at chemical introduction valves 408a, 408b. The chemical introduction valves 408a, 408b can be controlled to enable or disable the introduction of the mixed chemical disinfectant to the plumbing fixture. In particular, when a chemical disinfection is carried out valves 402a and 402b are closed to prevent water being introduced to the plumbing fixture, allowing the chemical disinfectant provided from the chemical holding tanks 404a, 404b to be distributed through the system. For example, when a chemical disinfection is to be applied, chemical introduction valves 408a, 408b may be opened to introduce chemicals into the plumbing system of the fixture 300 where the chemicals may be held for a period of time to disinfect the plumbing fixture. After holding for a period of time, the chemicals may be flushed through the plumbing systems, followed by a water flush to ensure that any chemicals which remain in the system are diluted so that a user may safely use the plumbing fixture 300. In some embodiments, at least one of the chemical holding tanks 404a, 404b may be configured to hold an ozone gas, which may be passed through the plumbing fixture to perform an ozone disinfection. In another embodiment, the plumbing fixture may further comprise an ozone generator which provides ozone gas to be passed through the plumbing fixture to perform an ozone disinfection. Preferably, the plumbing fixture is drained of water before an ozone disinfection is performed.

After passing through the valves 402a, 402b and chemical introduction valves 408a, 408b, the hot and cold water passes through water filters 410a, 410b which ensure that water supplying the plumbing fixture is clean and free from potentially contaminating impurities.

A second countermeasure device is present in the form of a metal ion system 412a, 412b which is present on each of the hot and cold water supplies. The metal ion system 412a, 412b provides ions such as silver and/or copper ions to each of the hot and cold water supplies, and the metal ions are effective to kill microorganisms which may be present in the water supply. In some examples the metal ion systems 412a, 412b may operate continuously such that metal ions are always present within the flow of water, or they may be activated only in response to a determined risk level related to biofilm formation as a countermeasure to counteract biofilm formation. Power for the metal ion system 412a, 412b is provided by two power supply units 413a, 413b.

After passing through the metal ion system 412a, 412b the hot water and cold water is mixed at a water valve 414 to be passed to a user, for example through a tap 308 as shown in FIG. 3. After flowing through the plumbing fixture, the waste water passes through a waste pipe 416 which leads directly to a plumbing system or waste water treatment system. The waste pipe 416 may comprise a flow meter to determine how much waste water passes therethrough. This information may be useful to determine if a user is introducing any foreign liquids to the plumbing fixture, which may present a contamination risk. This information may be used in a method as described above with respect to FIGS. 1 and 2. The waste pipe 416 may also comprise a valve which may be closed in order to retain a chemical disinfectant and/or water within the basin 302 and the waste pipe 41E for a predetermined period of time in order to disinfect the plumbing fixture 300 and/or remove biofilm.

The plumbing fixture further comprises a radio-frequency identification (RFID) detector 418 which may be used to identify a user and track the user to ensure compliance with a best-practice procedure for using the plumbing fixture, e.g. proper handwashing procedure. In some examples, the RFID detector 418 may be used to identify the last user of the plumbing fixture before a problem is detected, and this identification may be recorded in order to see patterns in users and any problems with the plumbing fixture. The RFID detector 418 may also, in some embodiments, be used to confirm that a user has permission to access the enclosure 400 to perform maintenance.

The plumbing fixture also comprises soap containers 420 and pumps 422 to allow automatic dispensing of soap and/or hand sanitizer to a user of the plumbing fixture. It is particularly preferred that the pumps 422 are peristaltic pumps, to reduce the risk of contamination. For example, a soap conduit may extend from the pumps 422 to a location near to the basin inlet such that soap and/or hand sanitizer can be dispensed into the basin, such as tube 316. In some embodiments, the plumbing fixture may further comprise a paper towel dispenser, for example a dispenser 328 as shown in FIG. 4.

At a lower side of the enclosure, the plumbing fixture comprises a drip tray 424 which is configured to catch any leaks within the enclosure, but may also be configured to monitor the amount of water within the drip tray 424 so that alerts may be sent to an operator if there is a significant level of leaking within the plumbing fixture. This information may also be used in a method as described above with respect to FIGS. 1 and 2. For example, if a leak has been detected it may automatically result in a high risk level being determined.

Although not shown, the enclosure 400 may comprise a number of UV lights or lamps which may be used to perform UV disinfection. For example, at least a portion of a water pipe within the enclosure 400 may be substantially clear to UV light, and a UV light may illuminate water passing through the pipe in order to disinfect the water flowing through the plumbing fixture. For example, the UV light may be switched on whenever water is flowing through the pipe. In some examples, UV lights may be provided within water pipes, or embedded into the sidewalls of water pipes, and/or may be provided to illuminate the basin 302 and/or any other surface of the plumbing fixture.

A processing system 426 (also referred to herein as a computing system) is also provided within the plumbing fixture. The processing system 426 is configured to control the components of the plumbing fixture, such as the valves and the countermeasure devices, and also configured to receive measurements from monitoring devices such as flow rate sensors, biofilm sensors or the like. In some embodiments, the processing system 426 may also be configured to communicate with a remote computing system, such as a central server, with which data may be shared or from which instructions may be received, for example. Additionally and/or alternatively, the processing system 426 may be configured to communicate with other plumbing fixtures, for example to share data such as determined risk levels or the like. The processing system 426 is thereby configured to carry out a method as described above with respect to FIGS. 1 and 2 to counteract biofilm formation within the plumbing fixture. In addition, the processing system 426 is configured to run automatic handwashing routines which may comprise controlling valves 402a, 402b to provide a stream of water at a particular temperature to a user and pumps 422 to provide soap and/or hand sanitizer, for example. The processing system 426 monitors information from the RFID detector 418 to monitor users of the plumbing fixture, which may ensure compliance with usage protocols and may also help monitor contamination risks posed by individual users. In some embodiments, the plumbing fixture may comprise a screen, for example screen 326 as shown in FIG. 4, which may be linked to the processing system 426 in order to display data to an operator and/or instructions to a user, for example. The processing system 426 may monitor supplies of chemicals in the holding tanks 404a, 404b as well as the level of soap within the containers 420. When levels of chemicals, soap and/or any other consumable associated with the plumbing fixture (e.g. paper towels) are low, the processing system 426 may provide an alert to an operator or user, for example by a screen or by communicating with a central server. In some examples, the processing system 426 may also track the age of each component within the plumbing fixture 300 and enclosure 400 to issue an alert such that components can be replaced when reaching the end of their rated lifespan.

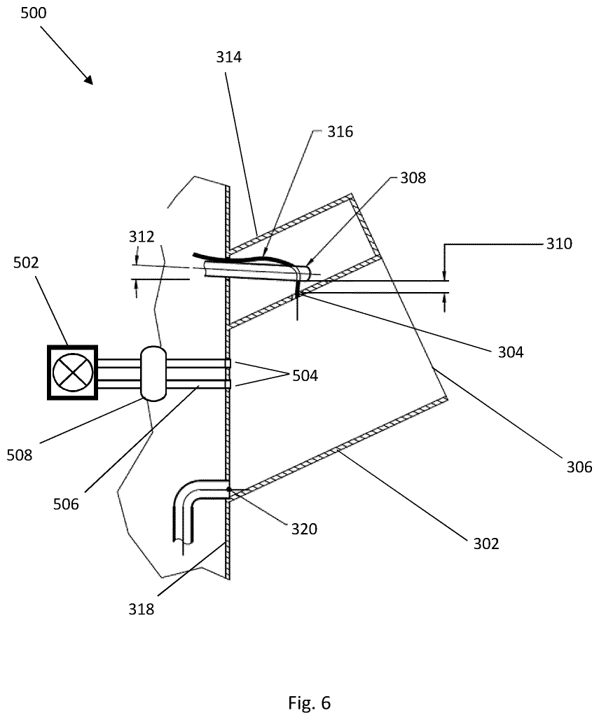

FIG. 6 shows a cross-section view of a plumbing fixture 500 which is a further embodiment of the present invention. In particular, the plumbing fixture 500 is a sink which is adapted to reduce contamination. Features of the plumbing fixture 500 which are the same as those discussed above with respect to FIG. 3 have been given the same reference numerals, and description thereof will not be repeated. Although not shown in FIG. 6 for ease of illustration, it should be understood that the longitudinal axis of the basin 302 of the plumbing fixture is disposed at an acute angle, as discussed above with respect to FIG. 3. The plumbing fixture 500 may be mounted to an enclosure substantially the same as enclosure 400, the contents of which are discussed in more detail above with respect to FIG. 5, such that a front surface of the enclosure provides a mounting surface 318 for the plumbing fixture 500. The plumbing fixture 500 may be used in a method as described above with respect to FIGS. 1 and 2, to enable monitoring of parameters relating to microbial presence and hence the determination of a level of risk related to biofilm formations within the plumbing fixture 500.

In addition to the features discussed above with respect to FIG. 3, the plumbing fixture 500 further comprises an air extraction system which is configured to generate a negative pressure within the basin 302 while the plumbing fixture is in use. In this way, air preferentially flows into the basin 302 and not out of the basin 302 such that aerosols and aerosolised particles do not escape the basin 302 while the plumbing fixture 500 is in use. In particular, the air extraction system is a filtered air extraction system as will be described below, as it is provided with a filtering device in the form of an air filter 508. By being provided with a filtered air extraction system, the plumbing fixture 500 is able to reduce or eliminate the emission of aerosolised particles from the basin 302 in use, wherein the aerosolised particles may comprise bio-aerosols comprising harmful bacteria or viruses which would otherwise be dispersed into the air around the plumbing fixture 500. For example, the aerosolised particles may be aerosolised water with bacteria, viruses or other pathogens, and/or biofilm. By preventing the emission of such bio-aerosols, the plumbing fixture 500 is able to help prevent the spread of harmful pathogens through air, in addition to reducing the spread of pathogens through water droplets as described above with respect to FIG. 3.

The filtered air extraction system comprises an extractor fan 502 which is arranged to draw air out of the basin 302 through air outlets 504 which are located in a rear wall of the basin 302 (e.g. located in the mounting surface 318). The extractor fan 502 is configured to generate an air flow rate of 22 litres per second when the plumbing fixture 500 is in use, though the air flow rate may of course be varied if necessary (for example, the air flow rate may depend on the size of the basin 302, with a larger basin 302 corresponding to an increased air flow rate). In some examples, there may be a single air outlet, or there may be two or more air outlets. Of course, it is envisaged that the air outlets 504 may be provided at any suitable location in the basin 302, but the outlets 504 are preferably spaced away from the opening 306 to ensure that the negative pressure is effective at ensuring no aerosols exit the basin 302. The extractor fan 302 may be configured to operate continuously, or may be configured to operate only when the plumbing fixture 500 is in use to wash a user's hands. When the extractor fan 502 is in operation, air is drawn out of the basin 302 through the air outlets 504, along air ducts 506, and through an air filter 508. The air filter 508 traps aerosolised particles which may be present in the air withdrawn from the basin 302, including bio-aerosols, to clean the air which is withdrawn from the basin. The filtered air continues along the air ducts 506 to the extractor fan 502, where the air may be dispersed, for example dispersed into the enclosure which houses the plumbing fixture 500, or dispersed elsewhere via additional air ducts.

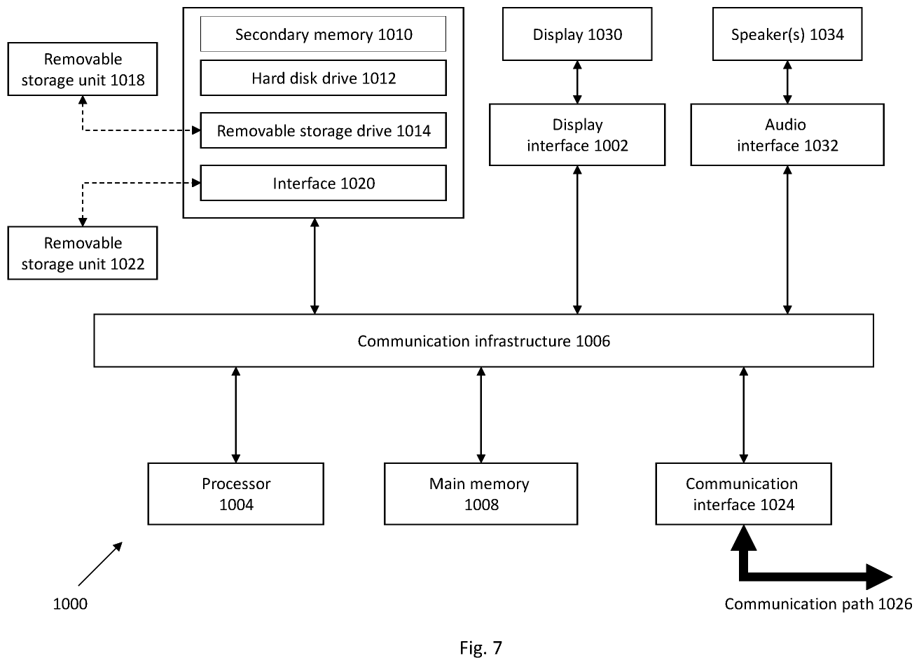

The air filter 508 preferably comprises a filter membrane (e.g. made of nylon, paper, or other suitable filter material) in a casing or housing (for example, a plastic housing which is configured to be connected to the air ducts 506. The filter 508 is chosen to ensure that aerosolised particles or droplets of 1 μm in diameter or larger are trapped by the filter b membrane, though of course it may be preferable to also capture and remove smaller particles from the air flow. A particularly preferred filter may be a Pall™ medical filter, for example an Ultipor® filter. The plumbing fixture 500 is preferably configured to monitor the number of times the plumbing fixture is used, and so may indicate (e.g. using a screen on the enclosure) when the filter needs to be changed. For example, the filter may be changed after a predetermined number of uses to ensure that the filtered air extraction system effectively removes aerosolised particles from the air withdrawn from the basin 302. In some examples, the filter 508 may be changed after a predetermined amount of air has been drawn through the filter 508, which may be determined by an elapsed operating time of the extractor fan 502, for example. This may be monitored by the system described above with respect to FIGS. 1, 2, 4 and 5. FIG. 7 depicts an example computing system 1000. The following description of the computing system 1000 is provided by way of example only and is not intended to be limiting.

The example computing system 1000 includes a processor 1004 for executing software routines. Although a single processor is shown for the sake of clarity, the computing system 1000 may also include a multi-processor system. The processor 1004 is connected to a communication infrastructure 1006 for communication with other components of the computing system 1000. The communication infrastructure 1006 may include, for example, a communications bus, cross-bar, or network.

The computing system 1000 further includes a main memory 1008, such as a random access memory (RAM), and a secondary memory 1010. The secondary memory 1010 may include, for example, a hard disk drive 1012 and/or a removable storage drive 1014, which may include a floppy disk drive, a magnetic tape drive, an optical disk drive, solid state storage or the like. The removable storage drive 1014 reads from and/or writes to a removable storage unit 1018 in a well-known manner. The removable storage unit 1018 may include a floppy disk, magnetic tape, optical disk, removable solid state storage (e.g. SD card) or the like, which is read by and written to by removable storage drive 1014. As will be appreciated by persons skilled in the relevant art(s), the removable storage unit 1018 includes a computer readable storage medium having stored therein computer executable program code instructions and/or data.

In an alternative implementation, the secondary memory 1010 may additionally or alternatively include other similar means for allowing computer programs or other instructions to be loaded into the computing system 1000. Such means can include, for example, a removable storage unit 1022 and an interface 1020. Examples of a removable storage unit 1022 and interface 1020 include a program cartridge and cartridge interface (such as that found in video game console devices), a removable memory chip (such as an EPROM or PROM) and associated socket, and other removable storage units 1022 and interfaces 1020 which allow software and data to be transferred from the removable storage unit 1022 to the computer system 1000.

The computing system 1000 also includes at least one communication interface 1024. The communication interface 1024 allows software and data to be transferred between computing system 1000 and external devices via a communication path 1026. In various embodiments, the communication interface 1024 permits data to be transferred between the computing system 1000 and a data communication network, such as a public data or private data communication network. The communication interface 1024 may be used to exchange data between a plurality of different computing systems 1000 that together form an interconnected computer network. Examples of a communication interface 1024 can include a modem, a network interface (such as an Ethernet card), a communication port, an antenna with associated circuitry and the like. The communication interface 1024 may be wired or may be wireless. Software and data transferred via the communication interface 1024 are in the form of signals which can be electronic, electromagnetic, optical or other signals capable of being received by communication interface 1024. These signals are provided to the communication interface via the communication path 1026.

As shown in FIG. 7, the computing system 1000 further includes a display interface 1002 which performs operations for rendering images to an associated display 1030 and an audio interface 1032 for performing operations for playing audio content via associated speaker(s) 1034.

As used herein, the term “computer program product” may refer, in part, to removable storage unit 1018, removable storage unit 1022, a hard disk installed in hard disk drive 1012, or a carrier wave carrying software over communication path 1026 (wireless link or cable) to communication interface 1024. A computer readable medium can include magnetic media, optical media, or other recordable media, or media that transmits a carrier wave or other signal. These computer program products are devices for providing software to the computing system 1000.

The computer programs (also called computer program code) are stored in main memory 1008 and/or secondary memory 1010. Computer programs can also be received via the communication interface 1024. Such computer programs, when executed, enable the computing system 1000 to perform one or more features of embodiments discussed herein. In various embodiments, the computer programs, when executed, enable the processor 1004 to perform features of the above-described embodiments. Accordingly, such computer programs represent controllers of the computer system 1000.

Software may be stored in a computer program product and loaded into the computing system 1000 using the removable storage drive 1014, the hard disk drive 1012, or the interface 1020. Alternatively, the computer program product may be downloaded to the computer system 1000 over the communications path 1026. The software, when executed by the processor 1004, causes the computing system 1000 to perform functions of embodiments described herein.

It is to be understood that the embodiment of FIG. 6 is presented merely by way of example. Therefore, in some embodiments one or more features of the computing system 1000 may be omitted. Also, in some embodiments, one or more features of the computing system 1000 may be combined together. Additionally, in some embodiments, one or more features of the computing system 1000 may be split into one or more component parts.

It will be appreciated that the elements illustrated in FIG. 7 function to provide means for performing the various functions and operations of the computer systems as described in the above embodiments. Although a few preferred embodiments have been shown and described, it will be appreciated by those skilled in the art that various changes and modifications might be made without departing from the scope of the invention, as defined in the appended claims.

All of the features disclosed in this specification (including any accompanying claims, abstract and drawings), and/or all of the steps of any method or process so disclosed, may be combined in any combination, except where at least some of such features and/or steps are mutually exclusive.

Each feature disclosed in this specification (including any accompanying claims, abstract and drawings) may be replaced by alternative features serving the same, equivalent or similar purposes, unless expressly stated otherwise. Thus, unless expressly stated otherwise, each feature disclose is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A computer implemented method for counteracting biofilm formation in a plumbing fixture, the method comprising:
monitoring a parameter relating to microbial presence within the plumbing fixture;
determining a level of risk related to biofilm formation based on the monitored parameter; and
in accordance with the determined level of risk, applying a countermeasure to the plumbing fixture in order to counteract biofilm formation.

2. A computer implemented method according to claim 1, further comprising storing information related to any one or more of:
the monitored parameter,
the determined level of risk, and/or
the countermeasure applied.

3. A computer implemented method according to claim 2, wherein determining a level of risk comprises analysing previously stored information.

4. A computer implemented method according to claim 2 or claim 3, wherein determining a level of risk comprises determining a trend in previously stored information.

5. A computer implemented method according to any one of claims 2 to 4, further comprising a step of analysing previously stored information to determine a countermeasure to apply to the plumbing fixture.

6. A computer implemented method according to any one of the preceding claims, wherein determining a level of risk comprises comparing the monitored parameter with a predetermined threshold.

7. A computer implemented method according to any one of the preceding claims, wherein the method further comprises receiving a level of risk related to biofilm formation associated with another plumbing fixture; and
applying a countermeasure to the plumbing fixture in accordance with the received level of risk.

8. A computer implemented method according to any one of the preceding claims, wherein the method further comprises receiving an instruction to apply a countermeasure to the plumbing fixture; and
applying a countermeasure to the plumbing fixture in accordance with the instruction.

9. A computer implemented method according to any one of the preceding claims, further comprising a step of adjusting a monitoring frequency.

10. A computer implemented method according to claim 9, wherein the monitoring frequency is determined according to the determined level of risk.

11. A computer implemented method according to any one of the preceding claims, wherein monitoring a parameter comprises monitoring any one or more of:
a hot water temperature,
a cold water temperature,
a water flow rate, and/or
a biofilm level.

12. A computer implemented method according to any one of the preceding claims, wherein applying a countermeasure comprises any one or more of:
flushing water through the plumbing fixture,
performing a disinfection
introducing metal ions to the water supply, and/or
disabling the plumbing fixture.

13. A computer implemented method according to claim 12, wherein performing a disinfection may comprise any one or more of:
performing a chemical disinfection,
performing ultraviolet (UV) light disinfection; and/or
performing an ozone disinfection.

14. A system for counteracting biofilm formation in a plumbing fixture, the system comprising:
a plumbing fixture;
a local computing system; and
a remote computing system, wherein
the local computing system and the remote server are configured to perform a method according to any one of claims 1 to 13.

15. A computer implemented method for counteracting biofilm formation in a plurality of plumbing fixtures, the method comprising:
monitoring, for each plumbing fixture, a parameter relating to microbial presence within that plumbing fixture;
determining, for each plumbing fixture, a level of risk related to biofilm formation based on the monitored parameter for that plumbing fixture; and
applying, for each plumbing fixture, a countermeasure in accordance with the determined level of risk for that plumbing fixture, in order to counteract biofilm formation.

16. A computer implemented method according to claim 15, wherein the plurality of plumbing fixtures are divided into a plurality of groups, and wherein the method further comprises:
in accordance with the determined level of risk for a first plumbing fixture, applying a countermeasure to each plumbing fixture within a group to which the first plumbing fixture belongs in order to counteract biofilm formation.

17. A computer implemented method according to claim 16, wherein each group has an associated location identifier, and wherein the level of risk related to biofilm formation for each plumbing fixture is determined according to the associated location identifier for the group to which each plumbing fixture belongs.

18. A system for counteracting biofilm formation in a plurality of plumbing fixtures, the system comprising:
a plurality of plumbing fixtures;
a plurality of local computing systems, wherein each local computing system is associated with a respective plumbing fixture; and
a remote computing system associated with the plurality of plumbing fixtures, wherein
the plurality of local computing systems and the remote computing system are configured to perform a method according to any one of claims 1 to 13 or 15 to 17.

19. A plumbing fixture for reducing contamination, the plumbing fixture comprising:
a basin affixable to a mounting surface at a proximal end;
a water inlet through which a stream of water may be introduced to the basin; and
a drain to allow water to flow out of the basin; wherein
the basin is an enclosure having an opening at a distal end to allow a user to access the stream of water, the water inlet being formed in an upper surface of the basin, and wherein
the water inlet and the basin are configured such that the entire stream of water is incident on an interior surface of the basin, wherein the interior surface is angled to reflect the entire stream of water away from the opening.

20. A plumbing fixture according to claim 19, wherein the basin is generally tubular and has a longitudinal axis, and wherein the basin is disposed such that the longitudinal axis is at an acute angle with respect to a vertical plane.

21. A plumbing fixture according to claim 19 or claim 20, wherein a tap for providing the stream of water is spaced away from the water inlet.

22. A plumbing fixture according to claim 20, wherein the tap has a proximal end and a distal end, and wherein the distal end of the tap is disposed below the proximal end.

23. A plumbing fixture according to claim 21 or claim 22, wherein the tap is configured to produce a generally planar stream of water.

24. A plumbing fixture according to any one of claims 21 to 23, wherein the basin further comprises a housing for the tap, wherein the housing has a curved upper exterior surface.

25. A plumbing fixture according to any one of claims 19 to 24, further comprising an air extraction system configured to generate negative pressure within the basin.

26. A plumbing fixture according to claim 25, wherein the air extraction system comprises a filtering device configured to remove aerosol particles from a flow of air extracted from the basin.

27. A plumbing fixture according to claim 26, wherein the filtering device is configured to remove aerosol particles having a diameter of at least 1 μm.

28. A plumbing fixture according to any one of claims 19 to 27, further comprising any one or more of:
a biofilm sensor;
a flow sensor at the water inlet;
a flow sensor at the drain; and/or
a water temperature sensor.

29. A plumbing fixture according to any one of claims 19 to 28, further comprising a countermeasure device.

30. A plumbing fixture according to claim 28 or claim 29, further comprising a computer system configured to control operation of the plumbing fixture.

31. A plumbing fixture according to claim 30, wherein the computer system is configured to carry out a method according to any one of claims 1 to 13.

32. A plumbing fixture according to any one of claims 19 to 31, further comprising a radio-frequency identification (RFID) sensor.

33. A plumbing fixture according to any one of claims 19 to 32, further comprising a proximity sensor.
